(19) United States
(12) Patent Application Publication
(10) Pub. No.: US 2019/0388127 A1
(43) Pub. Date: Dec. 26, 2019

(54) Interpedicular Spacer

(71) Applicant: Lawrence P. Maccree, Knoxville, TN (US)

(72) Inventor: Lawrence P. Maccree, Knoxville, TN (US)

(21) Appl. No.: 16/438,748

(22) Filed: Jun. 12, 2019

Related U.S. Application Data
(60) Provisional application No. 62/688,417, filed on Jun. 22, 2018.

(51) Int. Cl. A61B 17/70; A61F 2/30

(52) U.S. Cl. CPC A61B 17/7064; A61B 17/8605; A61F 2002/3082; A61F 2/30771

(57) ABSTRACT

An interpedicular spacer having a top surface, two opposing end surfaces disposed at distal ends of the top surface, an arched lower surface opposing the top surface, and two screw holes, one each of the screw holes disposed so as to enter the interpedicular spacer through one of the distal ends of the top surface and exit the interpedicular spacer through one of the opposing end surfaces. The spacer is screwed into the pedicles of two adjacent vertebrae after a complete facetectomy, and does not extend out past the posterior margins of the facet complex.

### FIELD

This application claims rights and priority on prior pending U.S. provisional patent application Ser. No. 62/688,417 filed Jun. 22, 2018 the entirety of the disclosure of which is incorporated herein by reference. This invention relates to the field of spinal implants. More particularly, this invention relates to an interpedicular spacer.

### INTRODUCTION

Spinal fusion is a procedure that is typically accomplished with a round bar of material that transverses the space posterior to the facet complex, which is comprised of the superior articular processes and the inferior articular process of two adjacent vertebrae, and which is secured through the facet or lateral to the facet complex at the junction of the transverse process and upper pedicle at two adjoining vertebrae. This is done by passing one screw through each pedicle into the vertebral body both above and below the facet complex. Each screw that passes through the adjacent pedicles attaches to the bar at a substantially ninety-degree angle, and therefore enters the underlying pedicle substantially to axially.

Unfortunately, the bar tends to extend out past the posterior edges of the facet complex, which can create complications and, because of its position posterior to the pedicles, is subject to a certain amount of rotational force over time.

What is needed, therefore, is a system that tends to reduce issues such as those described above, at least in part.

### SUMMARY

The above and other needs are met by an interpedicular spacer having a top surface, two opposing end surfaces disposed at distal ends of the top surface, an arched lower surface opposing the top surface, and two screw holes, one each of the screw holes disposed so as to enter the interpedicular spacer through one of the distal ends of the top surface and exit the interpedicular spacer through one of the opposing end surfaces. The spacer is screwed into the pedicles of two adjacent vertebrae after a complete facetectomy, and does not extend out past the posterior margins of the facet complex.

In some embodiments according to this aspect of the invention, the two opposing end surfaces have a generally concave shape. In some embodiments, the top surface has a generally concave shape. In some embodiments, the two screw holes have smooth sidewalls. In some embodiments, the two screw holes have threaded sidewalls. In some embodiments, the two screw holes are centered along a common plane. In some embodiments, at least one surface of the interpedicular spacer is smooth. In some embodiments, at least one surface of the interpedicular spacer is textured. In some embodiments, at least one surface of the interpedicular spacer is ribbed.

According to another aspect of the invention there is described an interpedicular spacer having a top surface with a generally concave shape. Two opposing end surfaces with generally concave shapes are disposed at distal ends of the top surface. An arched lower surface opposes the top surface. Two smooth-walled screw holes are disposed so as to enter the interpedicular spacer through one of the distal ends of the top surface and exit the interpedicular spacer through one of the opposing end surfaces.

According to yet another aspect of the invention there is described a method of providing fixation between two adjacent vertebrae. An inferior articular process is substantially removed on a first side of a superior one of the adjacent vertebrae, thereby exposing an inferior face of a superior pedicle on the superior vertebra. A superior articular process is substantially removed on the first side of an inferior one of the adjacent vertebrae, thereby exposing a superior face of an inferior pedicle on the inferior vertebra. An interpedicular spacer is provided, which interpedicular spacer has, a top surface, two opposing end surfaces disposed at distal ends of the top surface, an arched lower surface opposing the top surface, and two screw holes, one each of the screw holes disposed so as to enter the interpedicular spacer through one of the distal ends of the top surface and exit the interpedicular spacer through one of the opposing end surfaces. The interpedicular spacer is disposed in a cavity created by the substantial removal of the superior and inferior articular processes, with one of the two opposing end surfaces disposed adjacent the inferior face of the superior pedicle and another of the two opposing end surfaces disposed adjacent the superior face of the inferior pedicle. A first screw is driven down through one of the screw holes, through the superior pedicle and into a body of the superior vertebra. A second screw is likewise driven down through another of the screw holes, through the inferior pedicle and into a body of the inferior vertebra.

In some embodiments according to this aspect of the invention, the two opposing end surfaces of the interpedicular spacer have a generally concave shape. In some embodiments, the top surface of the interpedicular spacer has a generally concave shape. In some embodiments, the two screw holes of the interpedicular spacer have smooth sidewalls. In some embodiments, the two screw holes of the interpedicular spacer have threaded sidewalls. In some embodiments, the two screw holes of the interpedicular spacer are centered along a common plane. In some embodiments, at least one surface of the interpedicular spacer is smooth. In some embodiments, at least one surface of the interpedicular spacer is textured. In some embodiments, at least one surface of the interpedicular spacer of the interpedicular spacer is ribbed.

### DRAWINGS

Further advantages of the invention are apparent by reference to the detailed description when considered in conjunction with the figures, which are not to scale so as to more clearly show the details, wherein like reference numbers indicate like elements throughout the several views, and wherein:

FIG. 1 depicts a first view of an interpedicular spacer according to an embodiment of the present invention.

FIG. 2 depicts a second view of an interpedicular spacer according to an embodiment of the present invention.

FIG. 3 depicts a third view of an interpedicular spacer according to an embodiment of the present invention.

FIG. 4 depicts a first view of a portion of a spinal column with an interpedicular spacer disposed between adjacent vertebrae, according to an embodiment of the present invention.

FIG. 5 depicts a second view of a portion of a spinal column with an interpedicular spacer disposed between adjacent vertebrae, according to an embodiment of the present invention.

FIG. 6 depicts a third view of a portion of a spinal column with an interpedicular spacer disposed between adjacent vertebrae, according to an embodiment of the present invention.

### DESCRIPTION

### Overview

The interpedicular spacer described herein fits between the two pedicles on one side of two adjacent vertebrae, after the pedicles have been exposed following a complete facetectomy, such as for decompression of the exiting nerve root. The interpedicular spacer provides stability between the adjacent vertebrae, which stability was previously provided by the facet complex that was removed, and may also support an anterior interbody fusion, whether that interbody fusion is performed from an anterior, lateral, transforaminal, or direct posterior approach. The interpedicular spacer is secured with screws that go through the interpedicular spacer and into the pedicles and the vertebral body to lock the interpedicular spacer into position and to stabilize the affected segment.

### Interpedicular Spacer

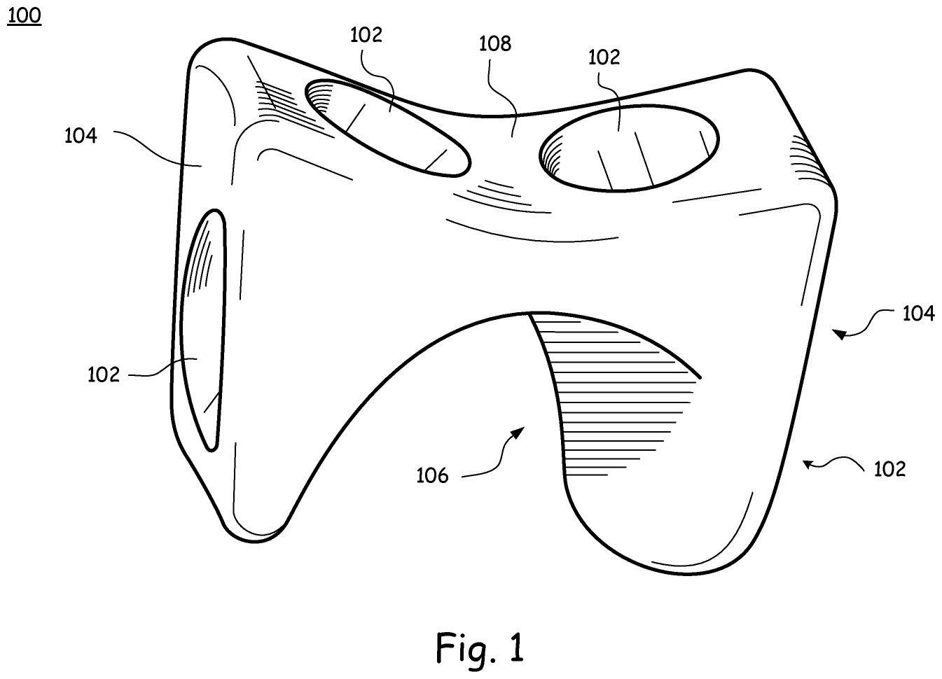

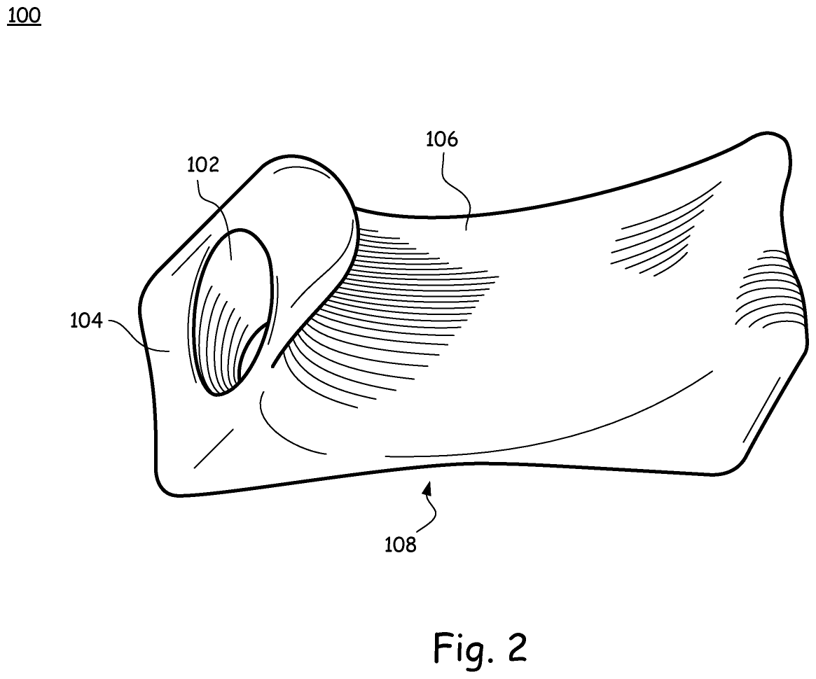

With reference now to FIGS. 1-3, there is depicted from several angles an interpedicular spacer 100 that fits between the pedicles of two adjacent vertebrae on a common side of the spinal column, and which has two concave end surfaces 104 that engage a substantial portion of the lateral cylindrical surface and posterior length of each pedicle. A posterior surface 108 of the interpedicular spacer 100 has a screw hole 102 at each end, near the two end surfaces 104. The screw holes 102 are formed at an angle such that a screw that enters the screw hole 102 through the entrance of one of the holes 102 at the posterior surface 108 emerges from the interpedicular spacer 100 through the exit of the hole 102 at one of the end surfaces 104 at an angle that is adapted to engage the side of the pedicle at that end of the interpedicular spacer 100. An arched or saddle-shaped bottom surface 106 provides space for the exiting nerve root.

Thus, the shape of the interpedicular spacer 100 acts to hold the interpedicular spacer 100 in place between the two pedicles, and the two screws hold the interpedicular spacer 100 tight to each pedicle. The screws are driven through each pedicle and into each associated vertebral body, giving each screw further contact area and thus sharing the stress load. In this manner, there is no portion of the interpedicular spacer 100 that extends past the posterior margins of the facet complex, and the screws enter each pedicle at an angle that tends to reduce or eliminate flexion, extension, side bending, and rotation of adjacent vertebrae, lessening the torque of the interpedicular spacer 100 and the two pedicles relative to one another.

The interpedicular spacer 100 can be fabricated in a number of different sizes, including different sizes and angles for all of the surfaces depicted, so as to better fit the patient. For example, and by way of explanation and not limitation, the interpedicular spacer 100 can be fabricated to have varying lengths, widths, heights, angles of end surfaces 104, height of the arched bottom surface 106, and radial curvature of end surfaces 104. In addition, the interpedicular spacer 100 can be fabricated of one or more of a number of different materials, such as metals, ceramics, and thermoformed materials.

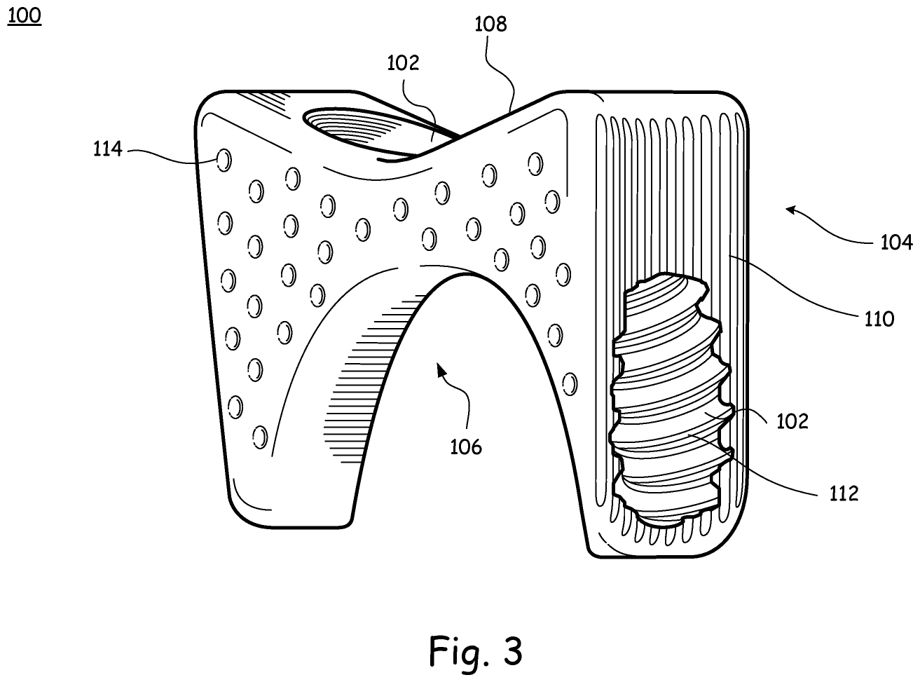

The surfaces of the interpedicular spacer 100 can be smooth, roughened to a greater or lesser extent, toothed, ribbed, and so forth, or a combination of one or more of the same. FIG. 3 depicts surfaces of the interpedicular spacer 100 that include ribs 110 and texture 114. The holes 102 for the screws can be formed at different angles, so as to give the screws purchase into the pedicles at higher or lower positions, or at steeper or shallower angles. Screw holes 102 and screws of different sizes are comprehended in the various embodiments of the invention. In some embodiments the screw holes 102 in the interpedicular spacer 100 have threads 112 at least in part, so as to engage the screws along the entire length of the screw holes 102, or along just a portion of the length of the screw holes 102. In other embodiments the sidewalls of the screw holes 102 are smooth, such that they do not substantially engage the threads of the screws passing therethrough.

### Interpedicular Spacer Positioning

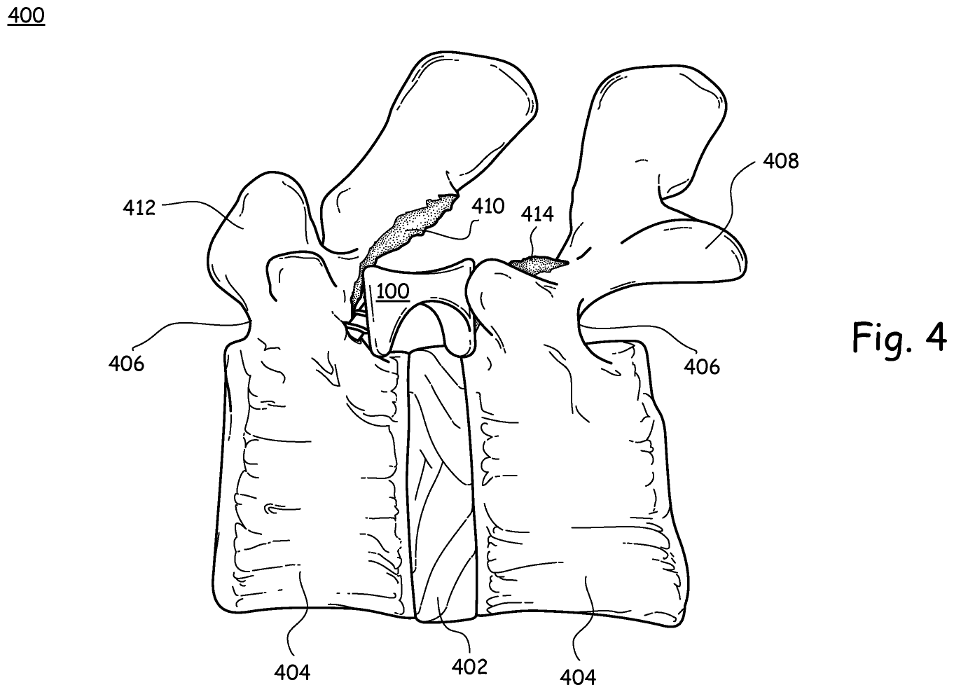

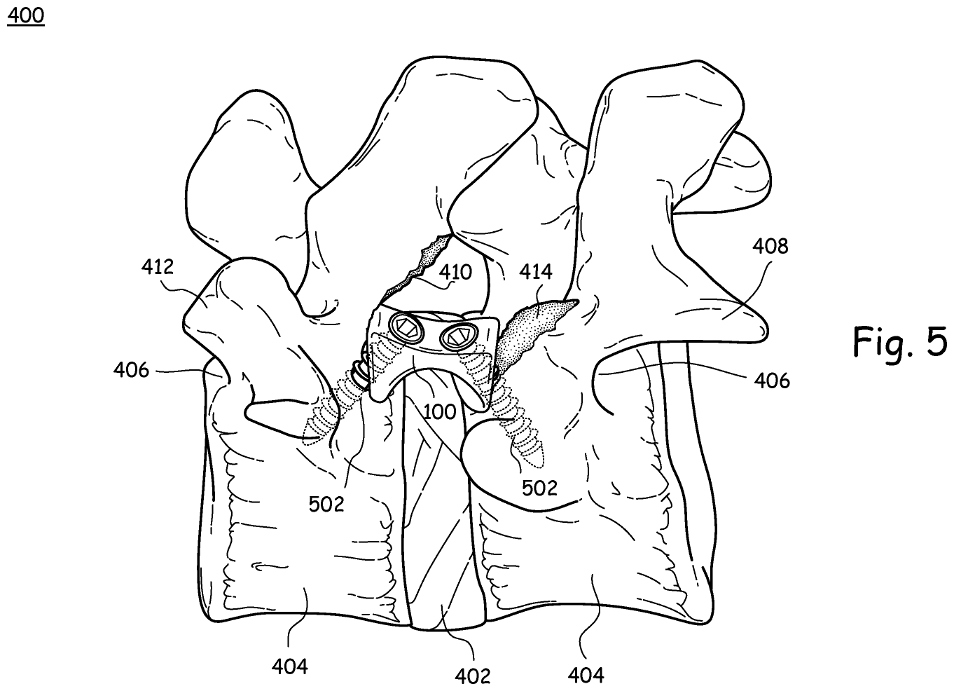

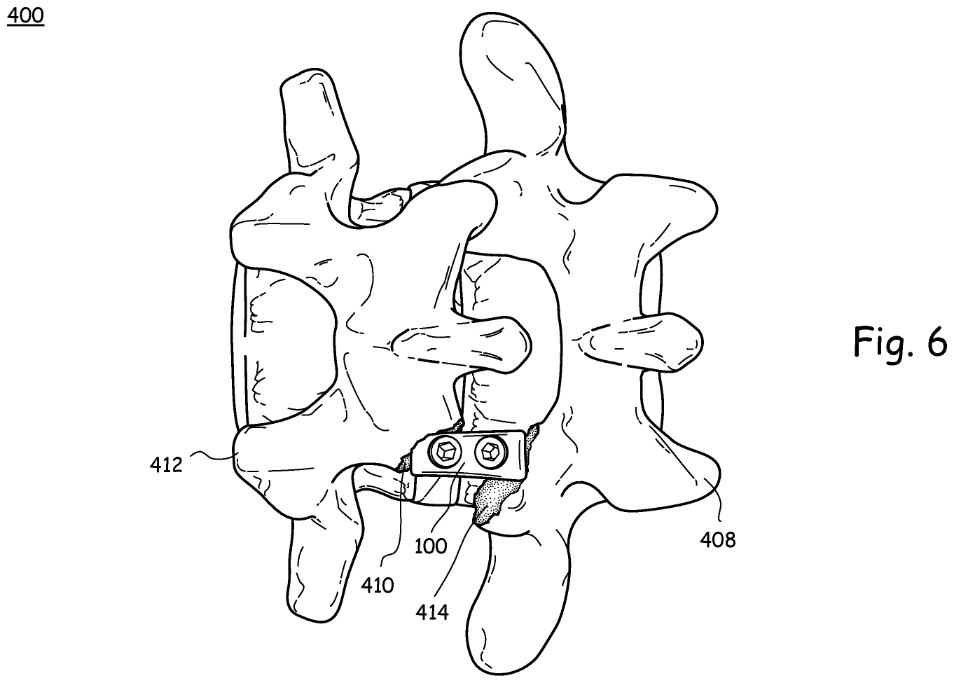

With reference now to FIGS. 4-6, there is shown from different angles the positioning of the interpedicular spacer 100 between adjacent vertebrae 404 of a spinal column. The interpedicular spacer 100 is inserted into place so as to substantially span an intervertebral disc 402 between two adjacent vertebrae 404 after a complete facetectomy. Specifically, the inferior articular process 408 of the superior vertebra 404 is substantially removed from the site 410, thereby exposing the inferior face of the pedicle 406, and the superior articular process 412 is substantially removed from the site 414 of the inferior vertebra 404, thereby exposing the superior face of the opposing pedicle 406.

The interpedicular spacer 100 is positioned such that the lower arched surface 106 is directed toward the intervertebral disc 402, and the two concave ends 104 of the interpedicular spacer 100 are disposed one each adjacent the two opposing pedicles 406. Screws 502 are then driven through the screw holes 102, such that they enter the pedicles 406 at an angle and travel down into the vertebral body.

The foregoing description of embodiments for this invention has been presented for purposes of illustration and description. It is not intended to be exhaustive or to limit the invention to the precise form disclosed. Obvious modifications or variations are possible in light of the above teachings. The embodiments are chosen and described in an effort to provide illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. An interpedicular spacer comprising:
a top surface,
two opposing end surfaces disposed at distal ends of the top surface,
an arched lower surface opposing the top surface, and
two screw holes, one each of the screw holes disposed so as to enter the interpedicular spacer through one of the distal ends of the top surface and exit the interpedicular spacer through one of the opposing end surfaces.

2. The interpedicular spacer of claim 1, wherein the two opposing end surfaces have a generally concave shape.

3. The interpedicular spacer of claim 1, wherein the top surface has a generally concave shape.

4. The interpedicular spacer of claim 1, wherein the two screw holes have smooth sidewalls.

5. The interpedicular spacer of claim 1, wherein the two screw holes have threaded sidewalls.

6. The interpedicular spacer of claim 1, wherein the two screw holes are centered along a common plane.

7. The interpedicular spacer of claim 1, wherein at least one surface of the interpedicular spacer is smooth.

8. The interpedicular spacer of claim 1, wherein at least one surface of the interpedicular spacer is textured.

9. The interpedicular spacer of claim 1, wherein at least one surface of the interpedicular spacer is ribbed.

10. An interpedicular spacer comprising:
a top surface having a generally concave shape,
two opposing end surfaces having generally concave shapes disposed at distal ends of the top surface,
an arched lower surface opposing the top surface, and
two smooth-walled screw holes, one each of the screw holes disposed so as to enter the interpedicular spacer through one of the distal ends of the top surface and exit the interpedicular spacer through one of the opposing end surfaces.

11. A method of providing fixation between two adjacent vertebrae, the method comprising the steps of:
substantially removing an inferior articular process on a first side of a superior one of the adjacent vertebrae, thereby exposing an inferior face of a superior pedicle on the superior vertebra,
substantially removing a superior articular process on the first side of an inferior one of the adjacent vertebrae, thereby exposing a superior face of an inferior pedicle on the inferior vertebra,
providing an interpedicular spacer having,
a top surface,
two opposing end surfaces disposed at distal ends of the top surface,
an arched lower surface opposing the top surface, and
two screw holes, one each of the screw holes disposed so as to enter the interpedicular spacer through one of the distal ends of the top surface and exit the interpedicular spacer through one of the opposing end surfaces,
disposing the interpedicular spacer in a cavity created by the substantial removal of the superior and inferior articular processes, with one of the two opposing end surfaces disposed adjacent the inferior face of the superior pedicle and another of the two opposing end surfaces disposed adjacent the superior face of the inferior pedicle,
driving a first screw down through one of the screw holes, through the superior pedicle and into a body of the superior vertebra, and
driving a second screw down through another of the screw holes, through the inferior pedicle and into a body of the inferior vertebra.

12. The method of claim 11, wherein the two opposing end surfaces of the interpedicular spacer have a generally concave shape.

13. The method of claim 11, wherein the top surface of the interpedicular spacer has a generally concave shape.

14. The method of claim 11, wherein the two screw holes of the interpedicular spacer have smooth sidewalls.

15. The method of claim 11, wherein the two screw holes of the interpedicular spacer have threaded sidewalls.

16. The method of claim 11, wherein the two screw holes of the interpedicular spacer are centered along a common plane.

17. The method of claim 11, wherein at least one surface of the interpedicular spacer is smooth.

18. The method of claim 11, wherein at least one surface of the interpedicular spacer is textured.

19. The method of claim 11, wherein at least one surface of the interpedicular spacer of the interpedicular spacer is ribbed.
